(19) United States
(12) Patent Application Publication
(10) Pub. No.: US 2011/0118666 A1
(43) Pub. Date: May 19, 2011

(54) Pressure infusion device with flexible window

(75) Inventors: Daniel M. Sivilich, Freehold, NJ (US); Paul Van der Heyden, Hillsborough, NJ (US)

(73) Assignee: Trimline Medical Products Corporation

(21) Appl. No.: 12/592,044

(22) Filed: Nov. 18, 2009

(51) Int. Cl. A61M 5/14

(52) U.S. Cl. 604/131

(57) ABSTRACT

The window material is made of two layers of material that have different elongation properties. The layers are bonded together to produce a multi-layer laminate that has sufficient optical clarity to easily read or scan the printed matter on the I.V. bag, has sufficient flexibility to provide maximum contact with the I.V. bag, but has little or no stretch to provide uniform pressure on the I.V. bag at typical utilization pressures. The preferred embodiment is a 10 mil, matte finished, high Durometer polyurethane film laminated to a 92 gauge (0.00092″) polyester film.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

The present invention relates to pressure infusion devices and more particularly to a pressure infusion device with a flexible window.

2. Description of Prior Art Including Information Disclosed Under 37 CFR 1.97 and 1.98

Various fluids for infusion into the body, such as normal saline, whole blood, or plasma, are normally supplied in disposable sterile sealed bags, typically called I.V. bags. An I.V. bag has an outlet port that is connected to a tube and catheter to supply the patient with the fluid.

Normally, I.V. bags are printed with the contents, directions for use and a scale indicating the volume of fluid being delivered. Further, hospitals often affix labels to I.V. bags with information such as the patient name and fluid dosage required. It is critical for the caregiver to be able to see the I.V. bag to verify the content and quantity of fluid administered in order to avoid mistakes as to what substances are provided to which patients and the quantity of the substance provided to the patient.

The I.V. bags are usually suspended above the patient and are designed to dispense the fluid at a rate dependent on gravity. However, in cases where the fluid must be delivered quickly and/or the pressure of the vessel into which the fluid is infused exceeds the pressure generated by gravity in a suspended I.V. bag, a pressure infusion device is employed. The pressure infusion device is inflated to pressurize the I.V. bag such that the desired rate of administration of the substance to the patient is achieved.

A typical pressure infusion device consists of primarily a bladder and a window which define a recess into which the I. V. is received. The window material must have relatively low extensibility, so that it does not stretch appreciably as the bladder is inflated. A means of inflating the bladder and a means of monitoring the pressure in the bladder are normally employed.

After the I.V. bag is situated in the recess formed between the bladder and window material, the pressure infusion device is inflated to a specific pressure, typically 300 mmHg. The system must maintain the specified pressure for sufficient time to administer the I.V. fluid. Accordingly, the bag and window material should not be elastic, so as to prevent “ballooning” and thus losses in pressure.

In order to prevent ballooning, the bladder is typically made of a low stretch fabric laminated to non-porous plastic film. The window material must also be substantially non-elastic but at the same time, it must have sufficient clarity to enable the caregiver to read or scan the labeling on the I.V. bag.

The two conventional methods used to obtain a window which is substantially non-elastic and at the same time sufficiently light transmissive of this purpose are to either use a mesh fabric as a window material, as described in the Bellin et. al. U.S. Pat. No. 4,735,613 or to use a rigid plastic transparent sheet as described in the Strobel et. al. U.S. Pat. No. 5,053,011.

Although the mesh window material has the desired low elongation characteristic, it has the disadvantage of reducing the readability and scanability of the I.V. bag. On the other hand, rigid or semi-rigid plastic windows have the preferred clarity, but are expensive, typically have high coefficients of friction on smooth surfaces such as the film of an I.V. bag, and can pose a hazard with sharp edges. Rigid materials also are resistant to wrapping around the I.V. bag and thus provide non-uniform pressure due to limited contact area with the I.V. bag.

It is, therefore, a prime object of the present invention to provide a pressure infusion device with a flexible window.

It is another object of the present invention to provide a pressure infusion device with a flexible window that is substantially non-elastic and at the same time highly light transmissive.

It is another object of the present invention to provide a pressure infusion device with a flexible window made of a laminate of a thermoplastic material and a low stretch material.

It is another object of the present invention to provide a pressure infusion device with a flexible window, wherein the window is made in part of a thermoplastic material such as polyurethane film.

It is another object of the present invention to provide a pressure infusion device with a flexible window, wherein the window is made in part of a low stretch material such as polyester.

It is another object of the present invention to provide a pressure infusion device with a flexible window, wherein the window is made of thermoplastic film and low stretch film bonded together by adhesive.

### BRIEF SUMMARY OF THE INVENTION

Those objects are achieved by the present invention which relates to a pressure infusion device including an inflatable bladder and a wall fixed to the bladder at remote locations so as to define a recess between the wall and the bladder. The wall includes a light transmissive window. The window is formed of a thermoplastic film and a low stretch film.

The thermoplastic film of the window is preferably made of polyurethane, most preferably contact clear urethane film. It is fixed directly to the bladder.

The thermoplastic film is preferably approximately 10 mils. thick, has a high Durometer (in the range of 85-90) and a matte finish.

The low stretch film is composed of polyester. The polyester film has approximately a 92 gauge and will not stretch more than 10% under the application of a stretching force of approximately 15 lbf of force.

The films are bonded together. Preferably the films are bonded together using a moisture-curable urethane-based adhesive.

The bonded films will not stretch more than 10% with under the application of a stretching force of approximately 17 lbf.

The wall is welded to the bladder.

The window preferably forms the entire wall.

The recess between the wall and the bladder is adapted to receive an I.V. bag therein. The bag is visible through the window.

In accordance with another aspect of the present invention, a pressure infusion device is provided including an inflatable bladder and a wall fixed to the bladder at remote locations so as to form a recess between the wall and the bladder. The wall includes a flexible, light transmissive window formed of a laminate of polyurethane.

The polyurethane is preferably a high Durometer polyurethane film approximately 10 mils. thick, with a matte finish and has approximately 92 gauge.

In accordance with another aspect of the present invention, a pressure infusion device is provided including an inflatable bladder and a wall fixed to said bladder at remote locations so as to define a recess between said wall and said bladder, said wall comprising a light transmissive window, said window comprising a high Durometer polyurethane film approximately 10 mils. thick bonded to a polyester film of approximately 92 gauge.

The polyurethane film has a matte finish.

The polyester film will not stretch more than 10% under the application of a stretching force of approximately 15 lbf of force.

The films are bonded together by a moisture-curable urethane-based adhesive.

The bonded films will not stretch more than 10% with under the application of a stretching force of approximately 17 lbf.

### BRIEF DESCRIPTION OF THE SEVERAL VIEWS OF DRAWINGS

To these and to such other objects that may hereinafter appears, the present invention relates to a pressure infusion device with a flexible window as described in detail in the following specification and recited in the annexed claims, taken together with the accompanying drawings, in which like numerals refer to like parts and in which:

FIG. 1 is a perspective view of the pressure infusion device of the present invention with an intravenous bag mounted therein;

FIG. 2 is a front elevation view of the pressure infusion device of the present invention;

FIG. 3 is a cross-sectional view of the pressure infusion device taken along line 3-3 of FIG. 2;

FIG. 4 is an enlarged sectional view of the portion of the pressure infusion device in the dashed circle on FIG. 3;

FIG. 5 is an exploded perspective view of the pressure infusion device showing the layers of the window.

### DETAILED DESCRIPTION OF THE INVENTION

The invention is a window material made of two or more layers of materials that have different elongation properties. They are bonded together by adhesive, heat, welding, or the like to produce a multi-layer laminate which can be used to form a window that has sufficient optical clarity to easily read or scan the labeling on the I.V. bag, has sufficient flexibility to provide maximum contact with the I.V. bag and at the same time has little or no elasticity so that it can provide uniform pressure on the I.V. bag at typical utilization pressures.

As seen in the drawings, the pressure infusion device of the present invention includes an inflatable bladder, generally designated A, designed for use with a conventional I. V. bag, generally designated B. Bladder A includes a front wall 10 and a rear wall 12. Walls 10 and 12 are sealed to each other along the periphery by any conventional means such as welding, except at spaced locations along the bottom through which tubes 14 and 16 extend. That configuration results in a closed member which can be inflated by pressurized gas such as air entering though tube 14 and be deflated by air exiting through that tube. A pressure gauge (not shown) is connected to tube 16.

Tube 14 would normally be connected through a valve (not shown) to a source of compressed air such as a hand pump (not shown). Once the pressure reaches the desired level, as indicated by the pressure gauge, the valve is closed and the pump removed. After the bag is empty, the valve is opened to allow the air in the bladder to exist through tube 14, thus deflating the bladder. By monitoring the pressure gauge connected to tube 16 and controlling the amount of pressurized air which enters the bladder through tube 14 in accordance with the gauge reading, the degree of inflation of bladder A, and hence the amount of pressure applied by bladder A on the I. V. bag B, can be carefully regulated.

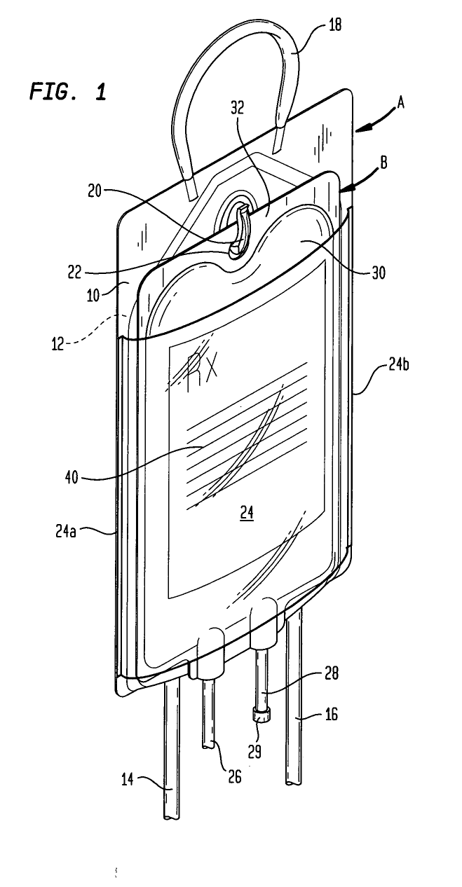

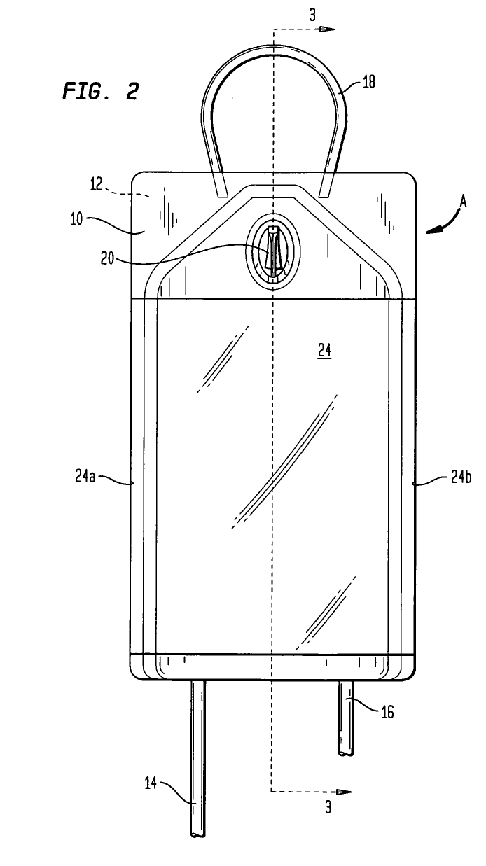

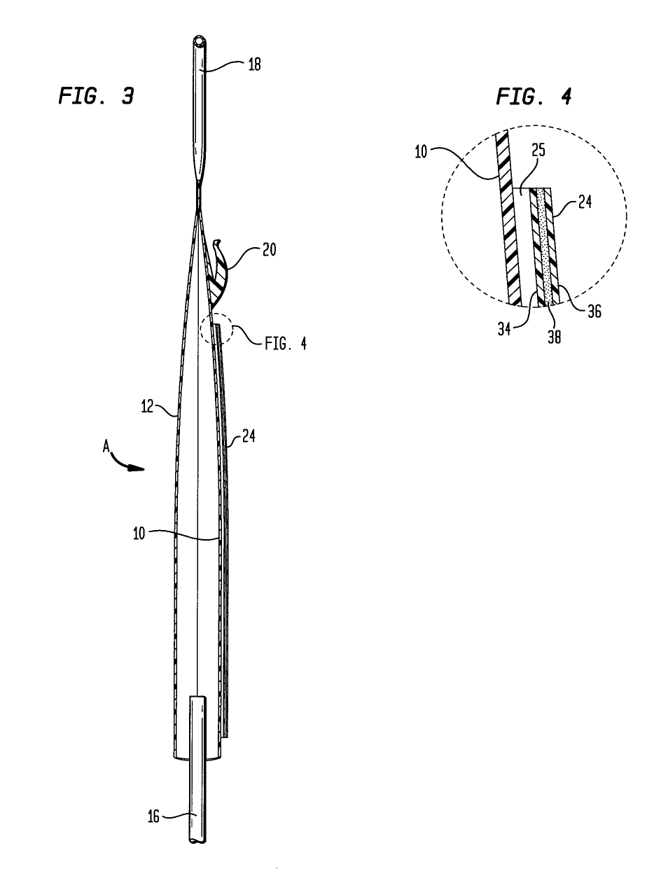

Bladder A has a hanger 18 extending from the top that permits the bladder to be hung on the branch of an I. V. pole. It also has a hook 20 extending outwardly from the upper portion of wall 10. Bag B is provided with an opening 22 spaced a short distance from the top edge designed to receive hook 20 so as to suspend bag B from bladder A, as seen in FIG. 1.

Bladder A also includes a separate wall formed by a light transmissive, flexible window 24. In the preferred embodiment, window 24 comprises the entire wall and has opposite side edges 24a and 24b sealed respectively to the side edges of bladder A so as to form a recess 25 between the exterior surface of wall 10 of bladder A and the interior surface of window 24. Recess 25 is adapted to receive the I. V. bag. The top and bottom of recess 25 are open such that the I. V. bag may be received in the recess when suspended from hook 20 and the fluid exit tube 26 and the air entrance tube 28 of the I. V. bag can extend out the bottom of the recess.

Bag B is formed of plastic sheets sealed to each other around the periphery to define a chamber 30 to contain the I. V. fluid. The fluid can exit the chamber through tube 26. The end of tube 28 is provided with a rubber cap 29 through which liquid may be injected by syringe into the fluid in the I. V. bag. The hook receiving opening 22 in the I. V. bag is spaced from the top edge of chamber 30 in a section 32. Section 32 of bag B is formed of both walls of the bag and thus has the increased strength necessary to allow the bag to hang properly within the bladder even when full.

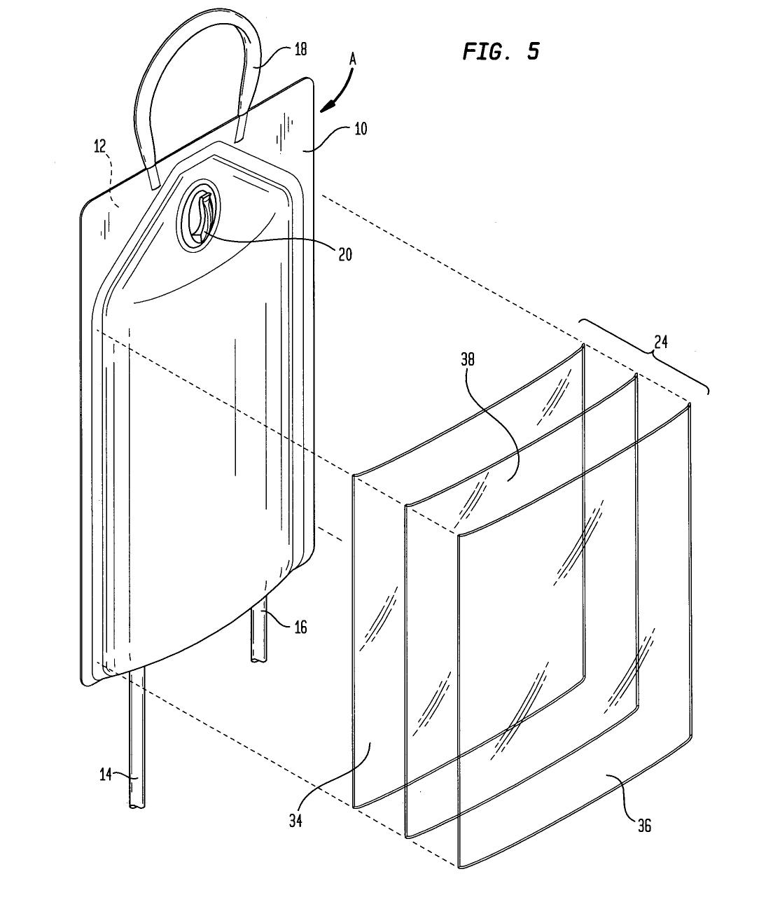

As best seen in FIG. 4 and FIG. 5, window 24 is formed of two plastic layers 34, 36 which are bonded together, preferably by a thin layer of adhesive 38. Layer 34 is formed of a thermoplastic film. Layer 36 is formed of a low stretch film, that is, a film with low elasticity such that it will expand only a limited amount when stretched.

The thermoplastic film side of window 24 is fixed to the side edges of the bladder by welding.

Preferably, thermoplastic film layer 34 is approximately 10 mils. thick, has a high Durometer value (within the range of 85 to 90 on a Shore A Durometer scale) and has a low friction matte finish.

Preferably, thermoplastic film layer 34 is formed of polyurethane. Most preferably, the film is formed of a contact clear urethane film.

Preferably, the low stretch film of layer 36 is formed of polyester, has a gauge of approximately 92 and will not stretch more than 10% under the application of a stretching force of approximately 15 lbf of force.

Preferably, the films are bonded together by a thin layer moisture-curable urethane-based adhesive 38.

When formed in this manner, the bonded films which make up the window will not stretch more than 10% with under the application of a stretching force of approximately 17 lbf. such that a uniform pressure will be applied to the I. V. bag as the bladder is inflated.

It is preferable that the window cover substantially the entire front wall of the bladder, from side edge to side edge, except the top portion from a point immediately below the hook and a small portion of the bottom, as illustrated.

It is important that the bag, and particularly the printed matter 40 on the surface of front wall 10, be is visible through the window such that the contents of the I. V. can be readily viewed and/or scanned by the caregiver. The materials and structure of the window are chosen with that objective in mind. However, the elasticity of the window is also critical in order to apply a uniform pressure on the bag as the bladder is inflated.

In selecting the materials for and the construction of the preferred embodiment of the invention, certain tests were preformed to optimize the light transmissive characteristics and low elasticity of the window to optimize the properties of the window.

In order to compare the elasticity of the window of the present invention with the fabric mesh component of the product disclosed in the aforementioned Bellin patent, a test was developed to use the Bellin fabric mesh component as a control and to measure the force required to stretch several test materials the same elongation as the control would stretch while in use in a pressure infusion device.

Accordingly, the following procedure was employed: The Vital Signs Infusable IN-9000 pressure infusor was selected as a control. A 125 mm ink line was marked on the mesh in the machine direction, which is the direction that a given fabric is produced and in which the majority of fibers are oriented. A 1000 ml bag of Lactated Ringer's Injection USP was inserted into the pressure infusor and the pressure increased to 300 mmHg as measured with an Omega digital pressure gauge. The length of the ink line was measured at several timed intervals. The increase in length was calculated as the base line deformation value.

The following measurements were made:

(a) Vital Signs IN-9000 mesh stretch under pressure.

| Time | Pressure (mm Hg) | Line Length (mm) |
|---|---|---|
| 10:50 AM | 0 | 125 |
| 10:56 AM | 303 | 136 |
| 12:03 PM | 302 | 136 |
| 03:48 PM | 304 | 136 |

Those measurements show an elongation of 8.8% for the fabric mesh component.

Samples of both the Vital Signs mesh (control) and the test materials were cut into 1″ cross direction×7″ machine direction strips using a steel rule die. Three strips of each sample were tested for machine direction tensile strength using a Zwick/Roell Z2.5 Tensile Tester. The force required to elongate the test and control samples 8.8% was measured. The samples were clamped into the Zwick jaws, which were 3″ apart. An increase of 8.8% is a calculated total travel of 3.264″ or a net travel of 0.264″ or 6.7 mm.

(b) The following average force values were obtained to stretch each sample 6.7 mm:

| | Force at 6.7 mm |
|---|---|
| Sample | Stretch (lbf) |
| Vital Signs scrim (Control) | 19.05 |
| 0.015″ contact clear urethane - film only | 5.23 |
| 0.010″ contact clear urethane - film only | 3.72 |
| 0.010″ High Durometer contact clear | 5.42 |
| urethane - film only | |
| 0.92 ga PET film - film only | 14.87 |
| 1.42 ga PET film - film only | 23.91 |
| 2.00 ga PET film - film only | 30.98 |
| 0.010″ urethane/0.92 ga PET film lab-made | 17.03 |
| laminate | |
| 0.010″ High Durometer urethane/92 ga PET | 19.68 |
| film lab-made laminate | |
| 0.010″ urethane/142 ga PET film lab-made | 25.06 |
| laminate | |

These results show that the tensile properties of the urethane and PET films are approximately additive in the desired maximum level of stretch. The laminates shown were bonded using a moisture-curable urethane-based adhesive.

The contact clear urethane film had a matte finish that had a lower coefficient of friction with the I.V. bag film than optical clear urethane samples that were investigated. Thus, it was easier to insert the I.V. bag into a pressure infusion device (a desirable feature) with a contact clear urethane window due to less frictional sticking than with an optical clear urethane.

Based upon the above, it was determined that the best window would be a window material made of two or more layers of materials that have different elongation properties. The layers should be bonded together by adhesive, heat, welding, or the like to produce a multi-layer laminate that has sufficient optical clarity to easily read or scan the labeling on the I.V. bag, have sufficient flexibility to provide maximum contact with the I.V. bag, but have little or no stretch to provide uniform pressure on the I.V. bag at typical utilization pressures.

It was determined that the preferred embodiment is a 10 mil, matte finished, high Durometer (in the range of 85 to 90) polyurethane film laminated to a 92 gauge (0.00092″) polyester film. This produces a flexible film with contact clarity and stretch characteristics comparable to a fabric mesh that is able to be welded to a pressure infusion device by radio frequency, able to conform to an I.V. bag to apply even pressure, and is cost effective.

While only a single preferred embodiment of the present invention has been disclosed for purposes of illustration, it is obvious that many modifications and variations could be made thereto. It is intended to cover all of those modifications and variations which fall within the scope of the present invention, as defined by the following claims.

1. A device for use with a flexible bag containing liquid to be infused into the body, the device comprising an inflatable bladder and a wall fixed to said bladder at remote locations so as to define a recess between said wall and said bladder into which the liquid containing bag is adapted to be received, such that said bladder applies pressure to said bag when inflated, said wall comprising a light transmissive window, said window comprising a thermoplastic film and a low stretch film.

2. The device of claim 1 wherein said thermoplastic film is fixed to said bladder.

3. The device of claim 1 wherein said thermoplastic film comprises polyurethane.

4. The device of claim 1 wherein said thermoplastic film is approximately 10 mils. thick.

5. The device of claim 1 wherein said thermoplastic film is high Durometer polyurethane film.

6. The device of claim 1 wherein said thermoplastic film has a matte finish.

7. The device of claim 3 wherein said polyurethane is contact clear urethane film.

8. The device of claim 1 wherein said low stretch film comprises polyester.

9. The device of claim 8 wherein said polyester film has approximately a 92 gauge.

10. The device of claim 9 wherein said polyester film will not stretch more than 10% under the application of a stretching force of approximately 15 lbf of force.

11. The device of claim 1 wherein said films are bonded together.

12. The device of claim 11 wherein said films are bonded together by a moisture-curable urethane-based adhesive.

13. The device of claim 11 wherein said bonded films will not stretch more than 10% with under the application of a stretching force of approximately 17 lbf.

14. The device of claim 1 wherein said wall is welded to said bladder.

15. The device of claim 1 wherein said window comprises said entire wall.

16. The device of claim 1 wherein the bag is an I.V. bag.

17. The device of claim 1 wherein said I. V. bag is visible through said window.

18. A device for use with a bag containing liquid for infusion into the body comprising an inflatable bladder and a wall fixed to said bladder at remote locations so as to form a recess between said wall and said bladder into which the liquid containing bag is adapted to be received, such that the bladder applies pressure to the bag when inflated, said wall comprising a flexible, light transmissive window, said window comprising a laminate of polyurethane.

19. The device of claim 18 wherein said polyurethane laminate comprises a high Durometer polyurethane film having a thickness of approximately 10 mils. and a matte finish.

20. The device of claim 18 wherein said laminate further comprises an approximately 92 gauge polyester film.

21. The device of claim 18 wherein said window will not stretch more than 10% under the application of a stretching force of approximately 17 lbf.

22. A device for use with a bag containing liquid for infusion into the body comprising an inflatable bladder and a wall fixed to said bladder at remote locations so as to define a recess between said wall and said bladder into which the liquid containing bag is adapted to be received, such that the bladder applies pressure to the bad when inflated, said wall comprising a window, said window comprising a high Durometer polyurethane film of a thickness of approximately 10 mils. bonded to an approximately 92 gauge polyester film.

23. The device of claim 22 wherein said polyurethane film has a matte finish.

24. The device of claim 22 wherein said polyester film will not stretch more than 10% under the application of a stretching force of approximately 15 lbf of force.

25. The device of claim 22 wherein said films are bonded together by a moisture-curable urethane-based adhesive.

26. The device of claim 22 wherein said bonded films will not stretch more than 10% with under the application of a stretching force of approximately 17 lbf.
